(19) United States
(12) Patent Application Publication
(10) Pub. No.: US 2012/0122148 A1
(43) Pub. Date: May 17, 2012

(54) Media For The Specific Detection Of Gram-Negative Bacteria Resistant To Beta-Lactam Antibiotics

(75) Inventor: Gilles Zambardi, Chezeneuve (FR)

(73) Assignee: bioMerieux, SA, Marcy L'Etoile (FR)

(21) Appl. No.: 13/386,460

(22) PCT Filed: Jul. 13, 2010
(86) PCT No.: PCT/FR2010/051474

§ 371 Date: Jan. 23, 2012

(30) Foreign Application Priority Data
Jul. 27, 2009 (FR) 09/03679

(51) Int. Cl. C12Q 1/18

(52) U.S. Cl. 435/32

(57) ABSTRACT

The invention relates to a reaction medium for gram-negative bacteria having a beta-lactam antibiotic resistance mechanism, comprising:
a marker for a beta-lactam antibiotic resistance mechanism, which is cefepime,
an inhibitor of a resistance mechanism other than said beta-lactam antibiotic resistance mechanism.

The field of the invention is that of microbiological analysis by means of biochemistry, and in particular the detection and identification of microorganisms, for instance bacteria or yeasts.

Bacterial resistance to antibiotics is a major public health problem. The resistance of infectious microorganisms to a treatment has developed at the same time as anti-infective molecules and today represents a major obstacle in therapeutics. This resistance is responsible for many problems, including difficulties in detection in the laboratory, limited treatment options and a deleterious impact on clinical outcome. In particular, the rapid and irrepressible increase in the resistance of pathogenic bacteria, over the last 20 years, represents one of the major current problems in medicine. Infections caused by these organisms are responsible for extended periods of hospitalization and are associated with high morbidity and mortality rates, following therapeutic failures.

Several resistance mechanisms can be involved simultaneously in a bacterial strain. They are generally classified in 3 categories: deficient penetration of the antibiotic into the bacterium, inactivation or excretion of the antibiotic by bacterial enzymatic systems, and lack of affinity between the bacterial target and the antibiotic.

Enzymatic inactivation is the most common mechanism of acquired resistance in terms of number of species and of antibiotics involved. Thus, chromosomal class C cephalosporinases today constitute one of the predominant resistance mechanisms of gram-negative bacteria, the bacteria expressing such enzymes being resistant to cephalosporins. Similarly, β-lactamases are enzymes expressed by certain bacteria, capable of hydrolyzing the C—N bond of the β-lactam ring, which is the basic structure of antibiotics of the β-lactam antibiotic family, so as to give a microbiologically inactive product. Several β-lactamase inhibitors (BLIs), such as clavulanic acid (CA), tazobactam and sulbactam, have been developed in order to increase the antimicrobial activity and broaden the spectrum of the β-lactam antibiotics which are associated therewith. They act as a suicide substrate for β-lactamases, and prevent enzymatic degradation of the antibiotics and allow them to become effective against bacteria that were initially resistant. However, by virtue of the persistent exposure of strains to antibiotic pressure, the bacteria express their ability to adapt through the continuous and dynamic production of β-lactamases, which evolves at the same time as the development of new molecules. Gram-negative bacteria which produce high-level chromosomal class C cephalosporinases (reference is made to HL Case bacteria), and also Gram-negative bacteria which produce extended-spectrum β-lactamases (reference is there made to ESBL bacteria) have, as a result, become an increasing threat, in particular because the number of bacterial species concerned is increasing. HL Case and ESBL bacteria are resistant to treatments based on 1st- and 2nd-generation cephalosporins and penicillins, but also on 3rd-generation cephalosporins (C3G) (cefotaxime CTX, ceftazidime CAZ, cefpodoxime CPD, ceftriaxone CRO) and monobactams (aztreonam ATM). On the other hand, 7α-methoxycephalosporins (cephamycins: cefoxitin, cefotetan) and carbapenems (imipenem, meropenem, ertapenem) generally retain their activity. ESBLs are inhibited by β-lactamase inhibitors (BLIs), which makes it possible to differentiate them from the other cephalosporinases.

These bacteria thus most commonly simultaneously express resistances to several treatments, which poses difficulties in setting up a relevant treatment and avoiding therapeutic failures. An Escherichia coli bacterium can thus be HL Case and ESBL. In addition, since ESBL-positive enterobacteria have a tendency to disseminate the resistance by clonal transmission of strains or conjugative plasmid transfer, they represent a problem in terms of controlling infections. In most studies, Escherichia coli and Klebsiella pneumoniae remain the most common ESBL-producing species. However, over the last few years, ESBLs have greatly broadened their panel of host species. Indeed, many species of enterobacteria and of nonfermenting gram-negative bacilli (such as Pseudomonas aeruginosa) have also been reported to be ESBL producers.

It therefore becomes essential, from a public health point of view, to be able to identify such microorganisms, and such resistance mechanisms, as rapidly as possible.

In general, the search for microorganisms resistant to a treatment is carried out according to the following steps:
1. taking a biological sample that may contain said microorganisms;
2. inoculating and incubating a culture medium (18 to 48 h) in order to induce exponential growth of the microorganisms;
3. pinpointing, on the culture media, colonies of potentially significant microorganisms;
4. characterizing the microorganism species;
5. identifying the mechanisms of resistance of the microorganisms analyzed, their biological significance and, optionally, the appropriate therapy.

This succession of steps involves a considerable amount of time between taking the sample that may contain the microorganisms and prescribing a treatment that is appropriate for the patient. Furthermore, the user must generally perform steps for transferring microorganisms from a first medium to a second medium manually, which can cause problems, in particular, of contamination, but also risks to the handler's health. By way of example, in order to detect the presence of extended-spectrum beta-lactamases (ESBLs) in strains of Escherichia coli and Klebsiella pneumoniae, use may be made of a diffusion technique as described in the publication by Jacoby & Han (J Clin Microbiol. 34(4): 908-11, 1996), which does not however give any information regarding the identification of the strains tested: it is possible to determine whether or not the bacterium is an ESBL-producing bacterium, but it is not possible to distinguish whether such a bacterium is an Escherichia coli or a Klebsiella pneumoniae.

Metabolic substrates are also used for detecting the presence of ESBLs or HL Cases. In this respect, AES laboratories proposes a medium in a biplate combining a Drigalski medium with cefotaxim and a MacConkey medium with ceftazidime. The Drigalski and MacConkey media make it possible to reveal lactose acidification, a metabolism which is present in a very large number of enterobacterial species. However, such a medium only makes it possible to distinguish resistant bacteria from non-resistant bacteria, and does not make it possible to distinguish bacteria expressing an ESBL from those expressing an HL Case. Neither does this medium make it possible to identify specific bacterial species, nor does it make it possible, for example, to discriminate between E. coli bacteria and K. pneumoniae bacteria.

In the case of the detection of resistance mechanisms other than ESBL, mention may be made of patent application EP0954560, which relates to the search for vancomycin-resistant enterococci, by combining vancomycin with a chromogenic medium that reveals two enzymatic activities (β-glucosidase and pyrrolidonyl-arylamidase). However, this chromogenic medium makes it possible to determine only whether or not the vancomycin-resistant strains belong to the Enterococcus genus, but does not make it possible to identify the species or the resistance mechanisms involved, in particular whether it is an acquired or wild-type resistance.

Thus, the characterization of a species of microorganism, and then the identification of its resistance to a treatment is lengthy and laborious. If the laboratory gives the clinician a positive screen when the isolate is in fact free of resistant microorganisms, this can lead to needless and inappropriate treatment. Conversely, not communicating a positive screen, which is subsequently confirmed, delays the setting up of the isolation of the patient (and possibly of an appropriate therapy) by one day. This shows the need for a rapid and reliable confirmation test.

The present invention therefore proposes to improve the prior art by providing a novel diagnostic tool which allows a gain in time, in reliability and in relevance with respect to the therapy implemented. Our invention makes it possible, in a single step, to identify the species of gram-negative microorganisms present in a sample and to determine their mechanism of resistance in order to propose a treatment appropriate to each patient.

Before going further in the disclosure of the invention, the following definitions are given in order to facilitate understanding of the invention.

The term “reaction medium” is intended to mean a medium comprising all the elements required for the survival and/or the growth of microorganisms, such as Staphylococcus aureus.

This reaction medium may either serve only as a revealing medium, or may serve as a culture and revealing medium. In the first case, the culturing of the microorganisms is carried out before inoculation and, in the second case, the reaction medium also constitutes the culture medium.

The reaction medium may be solid, semi-solid or liquid. The term “solid medium” is intended to mean for example, a gelled medium. Preferentially, the medium according to the invention is a gelled medium. Agar is the conventional gelling agent in microbiology for culturing microorganisms, but it is possible to use gelatin or agarose. A certain number of preparations are commercially available, for instance Columbia agar, Trypcase-soy agar, MacConkey agar, Sabouraud agar or more generally those described in the Handbook of Microbiological Media (CRC Press).

The reaction medium according to the invention may contain optional other additives, for instance: peptones, one or more growth factors, carbohydrates, one or more selective agents, buffer solutions, one or more gelling agents, etc. This reaction medium may be in the form of a liquid or of a gel that is ready to use, i.e. ready for inoculation in a tube or a flask, or on a Petri dish. When it is provided in the form of a gel in a flask, a prior regeneration (passage at 100° C.) of the medium is preferentially carried out before pouring into Petri dishes. It can also be a medium in powder form or in a flask which, before being poured into Petri dishes, tubes or flasks, has a supplement added thereto. Preferentially, the medium according to the invention is a selective medium, i.e. a medium comprising compounds which promote the growth of Gram-negative bacteria. Mention may in particular be made of sodium citrate, sodium sulfite, antibiotics such as vancomycin, des antifungals such as amphotericin B, natamycin or cycloheximide, surfactants such as bile salts, sodium deoxycholate or Tergitols, and dyes such as brilliant green, crystal violet, fuchsin, eosin or methylene blue. Preferentially, the medium according to the invention is a selective medium comprising compounds which promote the growth of extended-spectrum beta-lactamase (ESBL) bacteria. Mention may in particular be made of the cephalosporins :
first-generation cephalosporins, such as: cefalexin, cefaloridine, cefalotin, cefazolin, cefadroxil, cefazedone, cefatrizine, cefapirin, cefradine, cefacetrile, cefrodaxine, ceftezole;
second-generation cephalosporins, such as: cefoxitin, cefuroxime, cefamandole, cefaclor, cefotetan, cefonicide, cefotiam, loracarbef, cefmetazole, cefprozil, ceforanide;
third-generation cephalosporins, such as: cefotaxime, ceftazidime, cefsulodine, ceftriaxone, cefmenoxime, latamoxef, ceftizoxime, cefixime, cefodizime, cefetamet, cefpiramide, cefoperazone, cefpodoxime, ceftibuten, cefdinir, cefditoren, ceftriaxone, cefoperazone, cefbuperazone;
fourth-generation cephalosporins, such as cefepime, cefpirome;

By way of gram-negative bacteria, mention may in particular be made of bacteria of the following genera: Pseudomonas, Escherichia, Salmonella, Shigella, Enterobacter, Klebsiella, Serratia, Proteus, Campylobacter, Haemophilus, Morganella, Vibrio, Yersinia, Acinetobacter, Branhamella, Neisseria, Burkholderia, Citrobacter, Hafnia, Edwardsiella, Aeromonas, Moraxella, Pasteurella, Providencia and Legionella.

The expression “beta-lactam antibiotic resistance mechanism” is intended to mean any type of device which allows a microorganism to render a treatment partially or completely ineffective on said microorganism, guaranteeing its survival, said device being related to the expression of an enzyme belonging to the extended-spectrum β-lactamase group, or of an enzyme belonging to the group of class C cephalosporinases expressed at a high level.

The expression “marker for a beta-lactam antibiotic resistance mechanism” is intended to mean a compound which makes it possible to demonstrate such a resistance mechanism, such as cefepime and salts thereof (Masuyoshi S. et al., 1989—“Comparison of the in vitro and in vivo antibacterial activities of cefepime (BMY-28142) with ceftazidime, cefuzonam, cefotaxime and cefmenoxime.”).

The expression “inhibitor of a resistance mechanism other than said beta-lactam antibiotic resistance mechanism” is intended to mean a compound which makes it possible to indirectly inhibit the growth of organisms developing a particular resistance, without inhibition of gram-negative bacteria expressing said beta-lactam antibiotic resistance mechanism, such as cloxacillin (Jack and Richmond, 1970—“A comparative study of eight distinct beta-lactamases synthesized by gram-negative bacteria.”) for the inhibition of class C cephalosporinases.

For the purposes of the present invention, the substrate of an enzymatic or metabolic activity is chosen from any substrate that can be hydrolyzed into a product that allows the direct or indirect detection of an enzymatic activity of a metabolism, such as, in particular, an osidase activity, preferentially a glucuronidase, glucosidase or galactosidase activity.

It may be a natural or synthetic substrate. The metabolism of the substrate causes a variation in the physicochemical properties of the reaction medium or of the cells of organisms. This variation can be detected by physicochemical methods, in particular optical methods by the eye of the operator or by means of spectrometric, electrical, magnetic, etc., instruments. Preferentially, it is a variation in optical properties, such as a modification of absorption, of fluorescence or of luminescence.

As chromogenic substrate, mention may in particular be made of substrates based on indoxyl, flavone, alizarin, acridine, esculetin, phenoxazine, nitrophenol, nitroaniline, naphthol, catechol, hydroxyquinoline, coumarin, aminophenol or dichloroaminophenol. Preferentially, the substrate(s) used in the present invention is (are) indoxyl-based.

As fluorescent substrate, mention may in particular be made of substrates based on umbelliferone or on coumarin, based on resorufine, phenoxazine, naphthol, naphtyhlamine, 2′-hydroxyphenyl-heterocycle or 2′-aminophenyl-heterocycle, or else based on fluorescein.

Preferentially, the substrate used in the present invention is 5-bromo-4-chloro-3-indoxyl-beta-D-glucopyranoside, preferentially in combination with 5-bromo-6-chloro-3-indoxyl-beta-D-galactopyranoside. Other possible substrates; beta-glucosidase substrates: 5-bromo-6-chloro-3-indoxyl-beta-glucoside; dihydroxyflavone-beta-glucoside; 3-hydroxyflavone-beta-glucoside, 3,4-cyclohexenoesculetin-beta-glucoside (no reference is made to 3,4-cyclopentenoesculetin-beta-glucoside?); 8-hydroxyquinoline-beta-glucoside; 5-bromo-4-chloro-3-indoxyl-N-methyl-beta-glucoside; 6-chloro-3-indoxyl-beta-glucoside; 5 -bromo-3-indoxyl-beta-glucoside; 5-iodo-3-indoxyl-beta-glucoside; 6-iodo-3-indoxyl-beta-glucoside; 6-fluoro-3-indoxyl-beta-glucoside; alizarin-beta-glucoside; nitrophenyl-beta-glucoside; 4-methylumbelliferyl-beta-glucoside; naphtholbenzein-beta-glucoside; indoxyl-N-methyl-beta-glucoside; naphthyl-beta-glucoside; aminophenyl-beta-glucoside; dichloroaminophenyl-beta-glucoside; beta-galactosidase substrates: 5-bromo-4-chloro-3-indoxyl-beta-galactoside; dihydroxy-flavone-beta-galactoside; 3,4-cyclohexenoesculetin-beta-galactoside; 8-hydroxy-quinoline-beta-galactoside; 5-bromo-4-chloro-3-indoxyl-N-methyl-beta-galactoside; 6-chloro-3-indoxyl-beta-galactoside; 5-bromo-3-indoxyl-beta-galactoside; 5-iodo-3-indoxyl-beta-galactoside; 6-fluoro-3-indoxyl-beta-galactoside; alizarin-beta-galactoside; nitrophenyl-beta-galactoside; 4-methylumbelliferyl-beta-galactoside; naphtholbenzein-beta-galactoside; indoxyl-N-methyl-beta-galactoside; naphthyl-beta-galactoside; aminophenyl-beta-galactoside; dichloroaminophenyl-beta-galactoside; beta-glucuronidase substrates: 5-bromo-6-chloro-3-indoxyl-beta-glucuronide; dihydroxyflavone-beta-glucuronide; 3,4-cyclohexenoesculetin-beta-glucuronide; 8-hydroxyquinoline-beta-glucuronide; 5-bromo-4-chloro-3-indoxyl-beta-glucuronide; 5-bromo-4-chloro-3-indoxyl-N-methyl-beta-glucuronide; 6-chloro-3-indoxyl-beta-glucuronide; 5-bromo-3-indoxyl-beta-glucuronide; 5-iodo-3-indoxyl-beta-glucuronide; 6-fluoro-3-indoxyl-beta-glucuronide; alizarin-beta-glucuronide; nitrophenyl-beta-glucuronide; 4-methylumbelliferyl-beta-glucuronide; naphtholbenzein-beta-glucuronide; indoxyl-N-methyl-beta-glucuronide; naphthyl-beta-glucuronide; aminophenyl-beta-glucuronide; dichloroaminophenyl-beta-glucuronide; alpha-glucosidase substrates, alpha-galactosidase substrates, esterase, in particular lipase or phosphatase substrates, cellobiosidase substrates, ribosidase substrates and hexosaminidase substrates.

The substrates of the invention can be used in a broad pH range, in particular between pH 5.5 and 10, preferentially between 6.5 and 10. When the medium according to the invention comprises one or more substrates for beta-glucosidase enzymatic activity, the concentration of substrate(s) is preferentially between 0.01 and 2 g/l, even more preferentially between 0.02 and 0.2 g/l, and advantageously it is between 0.05 and 0.15 g/l. This is because, at this concentration of substrate, a better color contrast is obtained.

Preferentially, said chromogenic substrate is chosen from a glucuronidase substrate, a beta-glucosidase substrate and a beta-galactosidase substrate.

The term “biological sample” is intended to mean a clinical sample, derived from a specimen of biological fluid, or a food sample, derived from any type of food. This sample may thus be liquid or solid and mention may be made, in a nonlimiting manner, of a clinical sample of blood, plasma, urine or feces, or of rectal, nose, throat, skin, wound or cerebrospinal fluid specimens, a food sample from water, from drinks such as milk or a fruit juice; from yogurt, from meat, from eggs, from vegetables, from mayonnaise, from cheese; from fish, etc., a food sample derived from an animal feed, such as, in particular, a sample derived from animal meals.

In this respect, the invention relates to a reaction medium for gram-negative bacteria having a beta-lactam antibiotic resistance mechanism, comprising:
a marker for a beta-lactam antibiotic resistance mechanism, which is cefepime,
an inhibitor of a resistance mechanism other than said beta-lactam antibiotic resistance mechanism.

According to one preferred embodiment of the invention, said inhibitor of a resistance mechanism is cloxacillin. Preferentially, the concentration of cloxacillin is between 0.05 and 1 g/l and more preferentially between 0.1 and 0.3 g/l. Advantageously, it is 0.2 g/l.

According to one preferred embodiment of the invention, the concentration of cefepime is between 0.05 and 1 mg/l and more preferentially between 0.10 and 0.5 mg/l.

Advantageously, it is 0.25 mg/l.

According to one preferred embodiment of the invention, the reaction medium also comprises a substrate for an enzymatic or metabolic acitivity, preferentially a chromogenic substrate.

According to one preferred embodiment of the invention, said chromogenic substrate is chosen from a glucuronidase substrate, a beta-glucosidase substrate and a beta-galactosidase substrate.

According to one preferred embodiment of the invention, the concentration of chromogenic substrate is between 0.02 and 2 g/l and more preferentially between 0.03 and 0.5 g/l. Advantageously, it is 0.1 g/l.

According to one preferred embodiment of the invention, said medium comprises a combination of at least two chromogenic substrates. According to a first embodiment, this combination comprises a glucuronidase substrate and a beta-glucosidase substrate.

According to a second embodiment, this combination comprises a beta-galactosidase substrate and a beta-glucosidase substrate.

The invention also relates to the use of a medium as defined above, for detecting gram-negative bacteria resistant to beta-lactam antibiotics, preferentially extended-spectrum beta-lactamase (ESBL) bacteria.

The invention also relates to a method for detecting gram-negative bacteria resistant to beta-lactam antibiotics, characterized in that it comprises the following steps:
1. providing a reaction medium as defined above,
2. inoculating the medium with a biological sample to be tested,
3. leaving to incubate, and
4. detecting the presence of gram-negative bacteria resistant to beta-lactam antibiotics.
   The incubation is preferentially carried out at a temperature between 30° C. and 42° C. The gram-negative bacteria resistant to beta-lactam antibiotics are preferentially detected by a specific glucuronidase, beta-glucosidase or beta-galactosidase activity which makes it possible to obtain colored or fluorescent colonies. The other species appear colorless or have a color or fluorescence that is different from that of the colonies of Gram-negative bacteria resistant to beta-lactam antibiotics.

The example below is given by way of explanation and is in no way limiting in nature. It will make it possible to understand the invention more clearly.

### EXAMPLE 1

Choice of strains: The inventors selected strains which make it possible to evaluate the activity of antibiotics with respect to Gram-negative species: enterobacteria and nonfermenting bacilli. In particular, ESBL-producing strains, high-level cephalosporinase-producing strains (HL Case) and strains without a particular resistance profile, termed wild-type strains, were used.

Preparation of media: The media tested were media composed of the peptone base of the ChromID CPS medium (bioMérieux ref 43541) to which were added, after autoclaving, in molten media, 300 mg/l cloxacillin and, for medium T: 4 mg/l cefpodoxime, for medium A: 0.25 mg/L of cefepime, for medium B: 3 mg/l of cefamandole and for medium C: 3 mg/l of cefuroxime.

Inoculation of media: The media are inoculated by carrying out a 3-quadrant streaking method using bacterial suspensions at 0.5 McF. The media are then incubated for 24 hours at 37° C.

Reading of media: The media are observed visually after 18 h and 24 hours of incubation, the growth density and also the colorations and coloration strengths are evaluated according to the scale below:
− or 0: absence of growth or of expression of enzymatic acitivity (i.e. no coloration)
+: weak growth, or enzymatic activity
++: very strong growth density or strong enzymatic acitivity (very strong coloration)

### Results:

| | | | | Cefpodoxime | | Cefepime | | Cefamandole | | Cefuroxime | |
|---|---|---|---|---|---|---|---|---|---|---|---|
| | | | | 4 mg/l | | 0.25 mg/l | | 3 mg/l | | 3 mg/l | |
| | | | Incubation | T | | A | | B | | C | |
| | | | time | Growth | Enz act. | Growth | Enz act. | Growth | Enz act. | Growth | Enz act. |
| Klebsiella oxytoca | 0105100 | HL case | 18 h | ++ | ++ | − | − | + | + | + | ++ |
| | | | 24 h | ++ | ++ | − | − | + | ++ | ++ | ++ |
| Proteus mirabilis | 0105105 | HL case | 18 h | − | − | − | − | − | − | − | − |
| | | | 24 h | − | − | − | − | − | − | − | − |
| Proteus mirabilis | 0105109 | HL case | 18 h | − | − | − | − | − | − | + | ++ |
| | | | 24 h | − | − | − | − | − | − | + | ++ |
| Enterobacter spp | 9306069 | HL case | 18 h | ++ | ++ | ++ | ++ | ++ | ++ | ++ | ++ |
| | | | 24 h | ++ | ++ | ++ | ++ | ++ | ++ | ++ | ++ |
| Morganella morganii | 9904104 | HL case | 18 h | − | − | − | − | − | − | − | − |
| | | | 24 h | − | − | − | − | − | − | − | − |
| Enterobacter spp | 0503037 | Wild-type | 18 h | ++ | ++ | − | − | ++ | ++ | ++ | ++ |
| | | | 24 h | ++ | ++ | − | − | ++ | ++ | ++ | ++ |
| Serratia fonticola | 8001047 | Wild-type | 18 h | + | + | − | − | + | + | ++ | ++ |
| | | | 24 h | + | ++ | − | − | + | ++ | ++ | ++ |
| Escherichia coli | 0411146 | ESBL | 18 h | ++ | ++ | ++ | ++ | ++ | ++ | ++ | ++ |
| | | | 24 h | ++ | ++ | ++ | ++ | ++ | ++ | ++ | ++ |
| Escherichia coli | 0505101 | ESBL | 18 h | + | ++ | ++ | ++ | ++ | ++ | ++ | ++ |
| | | | 24 h | ++ | ++ | ++ | ++ | ++ | ++ | ++ | ++ |
| Klebsiella oxytoca | 0502094 | ESBL | 18 h | ++ | ++ | ++ | ++ | ++ | ++ | ++ | ++ |
| | | | 24 h | ++ | ++ | ++ | ++ | ++ | ++ | ++ | ++ |
| Enterobacter aerogenes | 0502112 | ESBL | 18 h | ++ | ++ | ++ | ++ | ++ | ++ | ++ | ++ |
| | | | 24 h | ++ | ++ | ++ | ++ | ++ | ++ | ++ | ++ |
| Proteus mirabilis | 9709068 | ESBL | 18 h | + | + | + | + | + | − | + | + |
| | | | 24 h | + | + | + | + | + | + | + | ++ |

### Conclusion:

The 4 molecules all enable good growth and expression of the enzymatic activities for the ESBL-producing strains. However, only cefepime enables good discrimination between the ESBL-producing strains, the strains producing an HL Case or the wild-type taxon strains. It is therefore the antibiotic which makes it possible to detect the ESBL strains with the best sensitivity and specificity.

1. A reaction medium for gram-negative bacteria having a beta-lactam antibiotic resistance mechanism, comprising:
a marker for a beta-lactam antibiotic resistance mechanism, which is cefepime,
an inhibitor of a resistance mechanism other than said beta-lactam antibiotic resistance mechanism.

2. The reaction medium as claimed in claim 1, wherein said inhibitor of a resistance mechanism is cloxacillin.

3. The reaction medium as claimed in claim 1, the concentration of cefepime is between 0.05 and 1 mg/l and more preferentially between 0.1 and 0.5 mg/l.

4. The reaction medium as claimed in claim 1, further comprising a substrate for an enzymatic or metabolic activity.

5. The reaction medium as claimed in claim 4, wherein the substrate is a chromogenic substrate is chosen from the group consisting of a glucuronidase substrate, a beta-glucosidase substrate and a beta-galactosidase substrate.

6. The use of a reaction medium as claimed in claim 1, for detecting gram-negative bacteria resistant to beta-lactam antibiotics.

7. The use of a reaction medium as claimed in claim 6, wherein the gram-negative bacteria resistant to beta-lactam antibiotics are extended-spectrum beta-lactamase (ESBL) bacteria.

8. A method for detecting gram-negative bacteria resistant to beta-lactam antibiotics, comprising the following steps:
a) providing a reaction medium as claimed in claim 1,
b) inoculating the medium with a biological sample to be tested,
c) leaving to incubate, and
detecting the presence of gram-negative bacteria resistant to beta-lactam antibiotics.

9. The reaction medium as claimed in claim 4, wherein said substrate for an enzymatic or metabolic activity is a chromogenic substrate.
